(19) United States
(12) Patent Application Publication
(10) Pub. No.: US 2010/0234614 A1
(43) Pub. Date: Sep. 16, 2010

(54) PROCESS FOR PURE IRBESARTAN

(75) Inventors: Bandi Parthasaradhi Reddy, Hyderabad (IN); Kura Rathnakar Reddy, Hyderabad (IN); Rapolu Raji Reddy, Andhrapradesh (IN); Dasari Muralidhara Reddy, Andrapradesh (IN); Matta Ramakrishna Reddy, Hyderabad (IN)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: HETERO DRUGS LIMITED, Hydrabad, Andhrapradesh (IN)

(21) Appl. No.: 12/306,721

(22) PCT Filed: Dec. 7, 2007
(86) PCT No.: PCT/IN2007/000569

§ 371 Date: Dec. 26, 2008

(51) Int. Cl. C07D 257/04; C07D 487/10

(52) U.S. Cl. 548/253; 548/300.7

(57) ABSTRACT

The present invention provides an improved and commercially viable process for preparation of irbesartan intermediate, 1-[(2′-cyanobiphenyl-4-yl)methyl]-2-n-butyl-4-spirocyclopentane-2-imidazolin-5-one, substantially free of 1-[(2′-cyanobiphenyl-4-yl)methyl]-2-n-propyl-4-spirocyclopentane-2-imidazolin-5-one impurity, thereby producing irbesartan substantially free of the undesired propyl analog impurity, namely 2-propyl-3-[[2′-(1H-tetrazol-5-yl)[1,1′-biphenyl]-4-yl]methyl]-1,3 -diazaspiro[4.4]non-1-en-4-one. The present invention also provides a process for preparation of irbesartan substantially free of tin content. The present invention further provides a commercially viable process for preparation of irbesartan in high purity and in high yield.

### FIELD OF THE INVENTION

The present invention provides an improved and commercially viable process for preparation of irbesartan intermediate, 1-[(2′-cyanobiphenyl-4-yl)methyl]-2-n-butyl-4-spirocyclopentane-2-imidazolin-5-one, substantially free of 1-[(2′-cyanobiphenyl-4-yl)methyl]-2-n-propyl-4-spirocyclopentane-2-imidazolin-5-one impurity, thereby producing irbesartan substantially free of the undesired propyl analog impurity, namely 2-propyl-3-[[2′-(1H-tetrazol-5-yl)[1,1-biphenyl]-4-yl]methyl]-1,3-diazaspiro[4.4]non-1-en-4-one. The present invention also provides a process for preparation of irbesartan substantially free of tin content.

The present invention further provides a commercially viable process for preparation of irbesartan in high purity and in high yield.

### BACKGROUND OF THE INVENTION

Irbesartan or 2-Butyl-3-[[2′-(1H-tetrazol-5-yl)[1,1′-biphenyl]-4-yl]methyl]-1,3-diazaspiro[4.4]non-1-en-4-one, which has the formula (1):

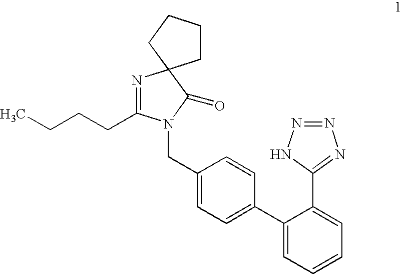

is a non-peptide angiotensin II—receptor antagonist.

The therapeutic uses of irbesartan and related compounds and their preparations are described in J. Med. Chem. 1993, 36, 3371-3380 disclosed in U.S. Pat. No. 5,270,317.

U.S. Pat. Nos. 5,270,317, 5,541,209 and 6,800,761 disclosed a process for preparation of irbesartan, which involves the condensation of a spiro compound with a halomethylbiphenyl compound in the presence of sodium hydride base and N,N-dimethylformamide solvent.

WO 99/38847 and WO2005/051943 describes a process for preparation of irbesartan, which involves the reaction of a Spiro compound with a halomethylbiphenyl compound in the presence of base and a phase transfer catalyst.

WO 2005/051943, WO 2005/122699 and WO 2006/001026 describes a process wherein the condensation of spiro compound and halomethylbiphenyl compound in the presence of a water miscible organic solvent and a base.

WO 2007/013101 describes a process for preparation of irbesartan, which involves the condensation of a spiro compound with a halomethylbiphenyl compound in the presence of mixture of solvents and base.

U.S. Pat. Nos. 5,270,317, 5,541,209 and 6,800,761 also disclosed the process for the preparation of tetrazole protected irbesartan from aromatic nitrile derivative in the presence of a protecting group, trialkyltin azide and o-xylene.

WO 2005/051943 and WO 20075/051929 disclosed the process for transformation of aromatic nitrile to tetrazole protected irbesartan in the presence of protecting group, trialkyl tin halide, metal azide and phase transfer catalyst.

WO 2007/013101 describes a process, which involves the treatment of the cyano compound with an azide in the presence of organic base and an organic acid optionally in the presence of aromatic and aliphatic hydrocarbons to yield irbesartan.

WO 2007/049293 and WO2007/052301 disclosed the process for the preparation of irbesartan from aromatic nitrile derivative in the presence of trialkyltin azide and o-xylene.

WO2006/023889 discloses a process for the preparation of compound of formula (1) from aromatic nitrile derivative in the presence of triethylaminechlorhydrate, sodium azide and N-methylpyrrolidinone.

WO2007/054965 discloses a process for the preparation of irbesartan from aromatic nitrile derivative in the presence of trialkyltinhalide, sodium azide, diisopropylethyl amine and o-xylene.

WO 2006/001026, WO 2007/020659, WO 2005/113518 and WO 2007/039117 described the preparation of irbesartan by treatment of cyano compound with trialkyltin halide, metal azide in xylene.

U.S. Pat. No. 5,629,331 describes a process for the conversion of cyano to tetrazole moiety in the presence of triethylamine hydrochloride, sodium azide and 1-methylpyrrolidin-2-one.

1-[(2′-Cyanobiphenyl-4-yl)methyl]-2-n-butyl-4-spirocyclopentane-2-imidazolin-5-one is the key intermediate in the preparation of irbesartan. The common impurity of the 1-[(2′-Cyanobiphenyl-4-yl)methyl]-2-n-butyl-4-spirocyclopentane-2-imidazolin-5-one is 1-[(2′-Cyanobiphenyl-4-yl)methyl]-2-n-propyl-4-spirocyclopentane-2-imidazolin-5-one. The 1-[(2′-Cyanobiphenyl-4-yl)methyl]-2-n-butyl-4-spirocyclopentane-2-imidazolin-5-one is prepared from valeric acid and usual impurity in the commercially available valeric acid is butyric acid and the 1-[(2′-Cyanobiphenyl-4-yl)methyl]-2-n-propyl-4-spirocyclopentane-2-imidazolin-5-one impurity in the 1-[(2′-Cyanobiphenyl-4-yl)methyl]-2-n-butyl-4-spirocyclopentane-2-imidazolin-5-one is due to the contamination of the starting material valeric acid with butyric acid. Since the commercial availability of valeric acid free of butyric acid is difficult, 1-[(2′-Cyanobiphenyl-4-yl)methyl]-2-n-butyl-4-spirocyclopentane-2-imidazolin-5-one is usually contaminated with 1-[(2′-Cyanobiphenyl-4-yl)methyl]-2-n-propyl-4-spirocyclopentane-2-imidazolin-5-one impurity. If such 1-[(2′-Cyanobiphenyl-4-yl)methyl]-2-n-butyl-4-spirocyclopentane-2-imidazolin-5-one is used for the preparation of irbesartan, the corresponding analog impurity, namely 2-Propyl-3-[[2′-(1H-tetrazol-5-yl)[1,1′-biphenyl]-4-yl]methyl]-1,3-diazaspiro[4.4]non-1-en-4-one impurity is also obtained along with irbesartan. The control of 2-Propyl-3-[[2′-(1H-tetrazol-5-yl)[1,1′-biphenyl]-4-yl]methyl]-1,3-diazaspiro[4.4]non-1-en-4-one impurity in the irbesartan is very difficult and many recrystallizations may require for reducing the content of 2-Propyl-3-[[2′-(1H-tetrazol-5-yl)[1,1′-biphenyl]-4-yl]methyl]-1,3-diazaspiro[4.4]non-1-en-4-one from irbesartan leading to heavy loss of the product yield. The present inventors have found a simple and effective process for the reduction of 1-[(2′-Cyanobiphenyl-4-yl)methyl]-2-n-propyl-4-spirocyclopentane-2-imidazolin-5-one impurity in the 1-[(2′-Cyanobiphenyl-4-yl)methyl]-2-n-butyl-4-spirocyclopentane-2-imidazolin-5-one intermediate. This process of the invention ensures the preparation of irbesartan substantially free of 2-Propyl-3-[[2′-(1H-tetrazol-5-yl)[1,1′-biphenyl]-4-yl]methyl]-1,3-diazaspiro[4.4]non-1-en-4-one impurity.

Irbesartan is prepared from 1-[(2′-Cyanobiphenyl-4-yl)methyl]-2-n-butyl-4-spirocyclopentane-2-imidazolin-5-one by reacting 1-[(2′-Cyanobiphenyl-4-yl)methyl]-2-n-butyl-4-spirocyclopentane-2-imidazolin-5-one with a tin compound such as tributyltin azide and subsequent deprotection and workup. Irbesartan obtained by the processes described in the art is found to contain tin in an amount above the limit of tin in irbesartan set as regulatory requirement. The inventors of the present invention have found a process for reducing the tin content in the irbesartan to the desired level.

One object of the present invention is to provide a process for preparation of irbesartan intermediate, 1-[(2′-cyanobiphenyl-4-yl)methyl]-2-n-butyl-4-spirocyclopentane-2-imidazolin-5-one, substantially free of 1-[(2′-cyanobiphenyl-4-yl)methyl]-2-n-propyl-4-spirocyclopentane-2-imidazolin-5-one, thereby producing irbesartan substantially free of the undesired propyl analog impurity, namely 2-propyl-3-[[2′-(1H-tetrazol-5-yl)[1,1′-biphenyl]-4-yl]methyl]-1,3-diazaspiro[4.4]non-1-en-4-one.

Another object of the present invention is to provide a process for preparation of irbesartan substantially free of tin content.

Another object of the present invention is to provide a process for preparation of irbesartan in high purity and in high yield.

### DETAILED DESCRIPTION OF THE INVENTION

According to one aspect of the present invention there is provided a process for preparation of 1-[(2′-Cyanobiphenyl-4-yl)methyl]-2-n-butyl-4-spirocyclopentane-2-imidazolin-5-one substantially free of 1-[(2′-Cyanobiphenyl-4-yl)methyl]-2-n-propyl-4-spirocyclopentane-2-imidazolin-5-one impurity, which comprises:
a) dissolving 1-[(2′-cyanobiphenyl-4-yl)methyl]-2-n-butyl-4-spirocyclopentane-2-imidazolin-5-one containing 1-[(2′-Cyanobiphenyl-4-yl)methyl]-2-n-propyl-4-spirocyclopentane-2-imidazolin-5-one impurity in an ester solvent;
b) adding an anti-solvent selected from an ether solvent and hydrocarbon solvent; or a mixture thereof; and
c) isolating the precipitated 1-[(2′-cyanobiphenyl-4-yl)methyl]-2-n-butyl-4-spirocyclopentane-2-imidazolin-5-one substantially free of 1-[(2′-Cyanobiphenyl-4-yl)methyl]-2-n-propyl-4-spirocyclopentane-2-imidazolin-5-one impurity.

The term “1-[(2-cyanobiphenyl-4-yl)methyl]-2-n-butyl-4-spirocyclo pentane-2-imidazolin-5-one substantially free of 1-[(2′-Cyanobiphenyl-4-yl)methyl]-2-n-propyl-4-spirocyclopentane-2-imidazolin-5-one impurity” refers to 1-[(2′-cyanobiphenyl-4-yl)methyl]-2-n-butyl-4-spirocyclopentane-2-imidazolin-5-one having the content of 1-[(2′-Cyanobiphenyl-4-yl)methyl]-2-n-propyl-4-spirocyclopentane-2-imidazolin-5-one impurity in less than about 0.2% by weight, preferably less than about 0.1% by weight and still more preferably less than about 0.05% by weight.

Preferable ester solvents used in step (a) are ethyl acetate and methyl acetate, more preferable being ethyl acetate.

Preferable ether anti solvents used in step (b) are diisopropyl ether, diethyl ether and methyl tertiarybutyl ether, and more preferable being diisopropyl ether.

Preferable hydrocarbon anti solvents used in step (b) are n-hexane and n-heptane and more preferable being n-hexane.

Isolation of 1-[(2′-cyanobiphenyl-4-yl)methyl]-2-n-butyl-4-spirocyclopentane-2-imidazolin-5-one substantially free of 1-[(2′-Cyanobiphenyl-4-yl)methyl]-2-n-propyl-4-spirocyclopentane-2-imidazolin-5-one impurity in step(c) may be carried out by the methods known such as filtration or centrifugation.

1-[(2′-cyanobiphenyl-4-yl)methyl]-2-n-butyl-4-spirocyclopentane-2-imidazolin-5-one substantially free of 1-[(2′-Cyanobiphenyl-4-yl)methyl]-2-n-propyl-4-spirocyclopentane-2-imidazolin-5-one impurity isolated may be converted to irbesartan by the methods known to obtain irbesartan substantially free of its propyl analog impurity namely, 2-propyl-3-[[2′-(1H-tetrazol-5-yl)[1,1′-biphenyl]-4-yl]methyl]-1,3-diazaspiro[4.4]non-1-en-4-one impurity.

According to another aspect of the present invention there is provided a process for the preparation of irbesartan substantially free of tin content the said process comprises:
a) stirring a solution or suspension of irbesartan containing tin content in a solvent selected from alcohol, ketone or water; or a mixture thereof in the presence of sulfuric acid at a pH below 1.5 for at least 20 minutes; and
b) isolating irbesartan at the same pH or at a different pH.

Preferably stirring in step (a) may be performed for 20 minutes to 3 hours, more preferably for 1 hour to 2 hours at a pH between 0.5 to 1.2.

Preferable alcohol solvents are methanol, ethanol and isopropyl alcohol, and more preferable solvents being methanol and ethanol.

Preferable ketone solvent is acetone.

Isolation of irbesartan in the step (b) may be carried out by crystallization, filtration or centrifugation; or a combination thereof.

“Irbesartan substantially free of tin content” refers to irbesartan containing tin content of 10 ppm or less, preferably less than 5 ppm, more preferably less than 2 ppm and still more preferably less than 1 ppm.

According to another aspect of the present invention, there is provided a process for preparation of highly pure irbesartan, which comprises:
a) reacting 1-[(2′-cyanobiphenyl-4-yl)methyl]-2-n-butyl-4-spirocyclopentane-2-imidazolin-5-one with tributyltin azide in xylene or toluene; and
b) treating the reaction mass obtained in step (a) with hydrogen chloride to obtain highly pure irbesartan,
   the said process is characterized in that the step(b) is carried out in the presence of ketonic solvent.

Preferable ketonic solvents are acetone, methyl ethyl ketone, methyl isobutyl ketone and diethyl ketone, and more preferable solvents being acetone and methyl isobutyl ketone.

Hydrogen chloride may be used in the reaction in step (b) directly in the form of a gas or hydrogen chloride gas dissolved in a ketonic solvent may be used. Preferably the reaction in step (b) may be carried out in the absence of water.

The reaction mass in step (b) may be stirred at least for about 2 hours, preferably stirred at least for about 3 hours and still more preferably stirred for about 3 hours to 4 hours.

After the reaction is completed, the reaction mass may then be subjected to usual work up such as washings, extractions etc.

The novel process provides irbesartan in high yield and purity, thus obviating the need to use column chromatography or additional purification steps to purify the material.

The following examples are given for the purpose of illustrating the present invention and should not be considered as limitations on the scope or spirit of the invention.

### Examples

### Example 1

### Step-I

2-n-Butyl-4-spirocyclopentane-2-imidazolin-5-one hydrochloride (1000 gm) and water are added to methylenedichloride (2700 ml) (pH 2.7 is observed). The solution is cooled to 20° C. and the pH is adjusted to 8.7 with 20% sodium carbonate solution. The layers are separated and the aqueous layer is extracted with methylenedichloride (4000 ml). Total organic layer is washed with water (4000 ml) and sodium chloride solution (2000 ml). Dried the mass, distilled off the solvent completely under vacuum, and then codistilled with acetone (1360 ml) to give 835 gm of 2-n-Butyl-4-spirocyclopentane-2-imidazolin-5-one (HPLC purity: 95.92%).

### Step-II

To the residue (obtained in step-I) is added acetone (8350 ml), potassium carbonate (1462 gm) and 4-Bromomethyl-2′-cyanobiphenyl (1136 gm). Tetra butyl ammonium bromide (42 gm) is added at 25-30° C. and the contents are heated to reflux for 5 hours (55° C.). Distilled off acetone completely and added water (5000 ml), methylene dichloride (5000 ml). The layers are separated and extracted with methylene dichloride (4000 ml). Total organic layer is washed with water (8000 ml) and 10% sodium chloride solution (4000 ml). Organic layer is dried, the solvent is distilled off completely under vacuum. And then codistilled with ethylacetate (600 ml) to give 1786 gm of 1-[(2′-Cyanobiphenyl-4-yl)methyl]-2-n-butyl-4-spirocyclopentane-2-imidazol in-5-one (HPLC purity: 91.74%, content of 1-[(2′-Cyanobiphenyl-4-yl)methyl]-2-n-propyl-4-spirocyclopentane-2-imidazolin-5-one impurity: 0.45%).

### Step-III

Ethyl acetate (1255 ml) is added to the residue (obtained in Step-II), the contents are heated to 50° C. until a clear solution is obtained. n-hexane (3700 ml) is added at 25-30° C., stirred for 15 minutes and cooled to 5° C. Stirred for 2 hours at 5° C. Filtered the solid and washed with a mixture of ethyl acetate: n-hexane (1:3) (1000 ml). Dried at 50° C. for 6 hours to give 1.43 kg of 1-[(2′-Cyanobiphenyl-4-yl)methyl]-2-n-butyl-4-spirocyclopentane-2-imidazolin-5-one (HPLC purity: 97.79%, content of 1-[(2′-Cyanobiphenyl-4-yl)methyl]-2-n-propyl-4-spirocyclopentane-2-imidazolin-5-one impurity: 0.05%).

### Example 2

### Step-I

2-n-Butyl-4-spirocyclopentane-2-imidazolin-5-one hydrochloride (500 gm), acetone (5000 ml), potassium hydroxide (360 gm) are stirred at 25-30° C. for 2 hours. Filtered on a cloth and separated the KCl salt. The filtrate is added to 4-Bromomethyl-2′-cyanobiphenyl (590 gm) and potassium carbonate at 25-30° C. The contents are heated to reflux for 5-6 hours at 53-56° C. Distilled off acetone completely and added water (2500 ml), methylene dichloride (2500 ml). The layers are separated and extracted with methylene dichloride (1400 ml). Total organic layer is washed with water (2800 ml) and 10% sodium chloride solution (1400 ml). Organic layer is dried, the solvent is distilled off completely under vacuum and then codistilled with ethylacetate (1200 ml) to give 770 gm of 1-[(2′-Cyanobiphenyl-4-yl)methyl]-2-n-butyl-4-spirocyclopentane-2-imidazolin-5-one (content of 1-[(2′-Cyanobiphenyl-4-yl)methyl]-2-n-propyl-4-spirocyclopentane-2-imidazolin-5-one impurity: 0.5%).

### Step-II

Ethyl acetate (600 Lt) is added to the residue (obtained in Step-I) and the contents are heated to 50° C. until a clear solution is obtained. To the solution n-hexane (1800 ml) is added at 25-30° C., stirred for 15 minutes and cooled to 5° C. Again stirred for 2 hours at 5° C. Filtered the solid and washed with n-hexane (1000 ml). Dried at 50° C. for 6 hours to give 670 gm of 1-[(2′-Cyanobiphenyl-4-yl)methyl]-2-n-butyl-4-spirocyclopentane-2-imidazolin-5-one (HPLC purity: 98.9, content of 1-[(2′-Cyanobiphenyl-4-yl)methyl]-2-n-propyl-4-spirocyclopentane-2-imidazolin-5-one impurity: 0.07%).

### Example 3

### Step-I

2-n-Butyl-4-spirocyclopentane-2-imidazolin-5-one hydrochloride (500 gm), acetone (5000 ml), potassium carbonate (1050 gm) are stirred at 25-30° C. for 2 hours to 2 hours 15 minutes. Filtered on a cloth, separated the KCl salt and carbonate, washed with fresh acetone (200 ml). To the filtrate 4-Bromomethyl-2′-cyanobiphenyl (590 gm), potassium carbonate (1050 gm) and tetra butyl ammonium bromide (25 gm) are added at 25-30° C. The contents are heated to reflux for 5-6 hours at 53-56° C. Distilled off acetone completely and added water (2500 ml), methylene dichloride (2500 ml). The layers are separated and extracted with methylene dichloride (1400 ml). Total organic layer is washed with water (2800 ml) and 10% sodium chloride solution (1400 ml). Organic layer is dried, the solvent is distilled off completely under vacuum and then codistilled with ethylacetate (1200 ml) to give 795 gm of 1-[(2′-Cyanobiphenyl-4-yl)methyl]-2-n-butyl-4-spirocyclopentane-2-imidazolin-5-one (content of 1-[(2′-Cyanobiphenyl-4-yl)methyl]-2-n-propyl-4-spirocyclopentane-2-imidazolin-5-one impurity: 0.48%).

### Step-II

Ethyl acetate (600 Lt) is added to the residue (obtained in Step-I) and the contents are heated to 50° C. until a clear solution is obtained. To the solution n-hexane (1800 ml) is added at 25-30° C., stirred for 15 minutes and cooled to 5° C. Again stirred for 2 hours at 5° C. Filtered the solid and washed with n-hexane (1000 ml). Dried at 50° C. for 6 hours to give 695 gm of 1-[(2′-Cyanobiphenyl-4-yl)methyl]-2-n-butyl-4-spirocyclopentane-2-imidazolin-5-one (HPLC purity: 98.06%, content of 1-[(2′-Cyanobiphenyl-4-yl)methyl]-2-n-propyl-4-spirocyclopentane-2-imidazolin-5-one impurity: 0.06%).

### Example 4

### Step-I

Sodium azide (300 gm), water are added to tributyl tin chloride for 1 hour to 2 hours at 0-5° C. Maintained for 2 hours at 0-5° C. o-Xylene (2000 ml) is added under stirring and the temperature is raised to 25-30° C. Maintained for 30 minutes to 45 minutes at 25-30° C. The layers are separated and the aqueous layer is extracted with o-Xylene (2000 ml). The total organic layer is washed with 10% sodium chloride (1500 ml). The organic layer is poured into 10 Lt reaction flask and added 1-[(2′-Cyanobiphenyl-4-yl)methyl]-2-n-butyl-4-spirocyclopentane-2-imidazolin-5-one (500 gm) under Dean and Starck apparatus set up. The contents are heated to reflux for 24-30 hrs at 145-148° C. Reaction mass is cooled to 25-27° C. and added acetone (2500 ml). The pH is adjusted to 2 by using acetone-HCl (Assay: 4.9%, Moisture content: 3%) (Acetone-HCl 2250 ml). The contents are stirred for 3 hours to 4 hours at 25-27° C., filtered the compound and washed with acetone (1000 ml) to give 542 gm of irbesartan (HPLC purity: 99.70%, 1-Pentanoylamino-cyclopentane carboxylic acid-[2′-(1H-tetrazole-5-yl)biphenyl-4-yl)methyliamide: 0.08%).

### Step-II

Irbesartan (obtained in Step-I) and water (5000 ml) are stirred for 15 to 30 minutes and added toluene (1500 ml). The pH is adjusted in between 11.0 and 11.5 at 20-25° C. by using 5% sodium hydroxide solution (1000 ml). The layers are separated and aqueous layer is washed with toluene (3000 ml). The aqueous layer is passed over hi-flow bed and washed with water (1000 ml). Initially the pH of the aqueous layer is adjusted between 3.0 to 3.5 and maintained for 15 to 20 minutes, again the pH is adjusted in between 0.5 to 1.0 using 1N sulfuric acid solution (920 ml). Stirred for 2 hours, filtered the compound and washed with water (5000 ml). Water (2500 ml) is added to the compound and the pH is re-adjusted in between 0.5 to 1.0 using 1N sulfuric acid solution (180-200 ml). Stirred for 2 hours at 25-30° C. to give 493 gm of irbesartan (HPLC purity: 97.02%, Tin content: 172.8ppm, 1-Pentanoylamino-cyclopentane carboxylic acid-[2′-(1H-tetrazole-5-yl)biphenyl-4-yl)methyl]amide: 2.32%).

### Step-III

Methylene dichloride (5000 ml) and methanol (500 ml) are added to Irbesartan (obtained in Step-II), washed with water (2500 ml) and the pH is adjusted to 3 by using 1N sulfuric acid solution (10 ml). The layers are separated and washed with water (2000 ml) at the pH 3. The organic layer is washed with sodium chloride solution (2000 ml), dried, distilled off the solvent completely and then codistilled with acetone (1500 ml). Acetone (2500 ml) is added to the compound and stirred for 30-45 minutes at 25-30° C. and maintained at 0-5° C. for 1 hour to 1 hour 15 minutes. The compound is filtered and washed with acetone (250 ml). Methanol (5000 ml) is added to the compound and heated to reflux (60-65° C.) till a clear solution is obtained. Added carbon, passed over a hi-flo bed and washed with hot methanol. Distilled off methanol and then codistilled with Acetone (1500 ml). Acetone (2500 ml) is added and stirred for 1 hour 30 minutes to 2 hours at 25-27° C., cooled to 0-5° C. and again stirred for 1 hour to 1 hour 30 minutes. Filtered the compound and washed with chilled acetone (400 ml) to give 430 gm of irbesartan (HPLC purity: 99.8%, Tin content: less than 1 ppm, 1-Pentanoylamino-cyclopentane carboxylic acid-[2′-(1H-tetrazole-5-yl)biphenyl-4-yl)methyl]amide: 0.04%).

What is claimed is:

1. A process for preparation of 1-[(2′-cyanobiphenyl-4-yl)methyl]-2-n-butyl-4-spirocyclopentane-2-imidazolin-5-one substantially free of 1-[(2′-cyanobiphenyl-4-yl)methyl]-2-n-propyl-4-spirocyclopentane-2-imidazolin-5-one impurity, which comprises:
a) dissolving 1-[(2′-cyanobiphenyl-4-yl)methyl]-2-n-butyl-4-spirocyclo pentane-2-imidazolin-5-one containing 1-[(2′-Cyanobiphenyl-4-yl)methyl]-2-n-propyl-4-spirocyclopentane-2-imidazolin-5-one impurity in an ester solvent;
b) adding an anti-solvent selected from an ether solvent and hydrocarbon solvent; or a mixture thereof; and
c) isolating the precipitated 1-[(2′-cyanobiphenyl-4-yl)methyl]-2-n-butyl-4-spirocyclopentane-2-imidazolin-5-one substantially free of 1-[(2′-Cyanobiphenyl-4-yl)methyl]-2-n-propyl-4-spirocyclopentane-2-imidazolin-5-one impurity.

2. The process as claimed in claim 1, wherein the ester solvent used in step (a) is ethyl acetate or methyl acetate.

3. The process as claimed in claim 2, wherein the ester solvent is ethyl acetate.

4. The process as claimed in claim 1, wherein the ether solvent used in step (b) is diisopropyl ether, diethyl ether or methyl tertiarybutyl ether.

5. The process as claimed in claim 4, wherein the ether solvent is diisopropyl ether.

6. The process as claimed in claim 1, wherein the hydrocarbon solvent used in step (b) is n-hexane or n-heptane.

7. The process as claimed in claim 6, wherein the hydrocarbon solvent is n-hexane.

8. The process as claimed in claim 1, wherein the isolation of 1-[(2′-cyanobiphenyl-4-yl)methyl]-2-n-butyl-4-spirocyclopentane-2-imidazolin-5-one substantially free of 1-[(2′-Cyanobiphenyl-4-yl)methyl]-2-n-propyl-4-spirocyclopentane-2-imidazolin-5-one impurity in step(c) is carried out by the methods such as filtration or centrifugation.

9. A process for preparation of irbesartan substantially free of tin content the said process comprises:
a) Stirring a solution or suspension of irbesartan containing tin content in a solvent selected from alcohol, ketone or water; or a mixture thereof in the presence of sulfuric acid at a pH below 1.5 for at least 20 minutes; and
b) isolating irbesartan at the same pH or at a different pH.

10. The process as claimed in claim 9, wherein the stirring in step (a) is performed for 20 minutes to 3 hours at a pH between 0.5 to 1.2.

11. The process as claimed in claim 10, wherein the stirring is performed for 1 hour to 2 hours at a pH between 0.5 to 1.2.

12. The process as claimed in claim 9, wherein the alcohol solvent used in step (a) is methanol, ethanol or isopropyl alcohol.

13. The process as claimed in claim 12, wherein the alcohol solvent is methanol or ethanol.

14. The process as claimed in claim 9, wherein the ketonic solvent used in step (a) is acetone.

15. The process as claimed in claim 9, wherein the isolation of irbesartan in step (b) is carried out by crystallization, filtration or centrifugation; or a combination thereof.

16. A process for preparation of highly pure irbesartan, which comprises:
a) reacting 1-[(2′-cyanobiphenyl-4-yl)methyl]-2-n-butyl-4-spirocyclopentane-2-imidazolin-5-one with tributyltin azide in xylene or toluene; and
b) treating the reaction mass obtained in step (a) with hydrogen chloride to obtain highly pure irbesartan,
the said process is characterized in that the step(b) is carried out in the presence of ketonic solvent.

17. The process as claimed in claim 16, wherein the ketonic solvent used in step (b) is acetone, methyl ethyl ketone, methyl isobutyl ketone or diethyl ketone.

18. The process as claimed in claim 17, wherein the ketonic solvent is acetone or methyl isobutyl ketone.

19. The process as claimed in claim 16, wherein the hydrogen chloride used in the reaction in step (b) is in the form of hydrogen chloride gas or hydrogen chloride gas dissolved in a ketonic solvent.

20. The process as claimed in claim 16, wherein the reaction in step (b) is carried out in the absence of water.
